(19) United States
(12) Patent Application Publication
(10) Pub. No.: US 2026/0240749 A1
(43) Pub. Date: Aug. 20, 2026

(54) COSMETIC WATER-IN-OIL FORMULATIONS COMPRISING SODIUM POLYACRYLATE, XANTHAN GUM AND POTASSIUM CETYL PHOSPHATE

(71) Applicant: Coty Inc., New York, NY (US)

(72) Inventors: Karine Barbe, MONACO (MC); Karine Julien, St André de la Roche (FR); Ornella Frisina, MONACO (MC)

(21) Appl. No.: 19/472,001

(22) PCT Filed: Mar. 1, 2024
(86) PCT No.: PCT/US2024/018240

§ 371 Date: Oct. 2, 2025

(30) Foreign Application Priority Data
Apr. 4, 2023 (FR) 2303319

(51) Int. Cl. A61K 8/81; A61K 8/06; A61K 8/55; A61K 8/99; A61Q 19/00

(52) U.S. Cl. CPC A61K 8/8141; A61K 8/064; A61K 8/55; A61K 8/99; A61Q 19/007; A61K 2800/48; A61K 2800/805

(57) ABSTRACT

Various examples disclosed relate to a cosmetic formulation including less than about 0.6 wt % gellant, but maintains a minimum viscosity of at least 2,000 cPs and is stable for up to four weeks after manufacture. The present disclosure provides a formulation having a gellant including both a polyacrylate and xanthan gum, an emulsifier capable of forming a water-in-oil formulation, one or more skin conditioning agents configured to treat a user's skin, and one or more solvents.

### CLAIM OF PRIORITY

This patent application claims the benefit of priority to French Application Serial No. FR2303319, filed Apr. 4, 2023, which is incorporated by reference herein in its entirety.

### BACKGROUND

Cosmetic formulations can be used to deliver any variety of beneficial effects to a user. Often the beneficial effect is provided by an active component. The ability to increase the diversity and amount of active component can increase the utility of the cosmetic formulation.

The cosmetics industry has increasing focus on skincare products and formulations. Various skincare products have been developed to address concerns of aging skin, wrinkles, elasticity, dryness, dullness, and other concerns. In particular, skin and face masks have become increasingly popular in treatment of skin for preservation and health of the face.

### SUMMARY OF THE DISCLOSURE

In an example, a cosmetic formulation includes: a gellant comprising a sodium polyacrylate and xanthan gum, wherein the gellant comprises less than about 0.8 wt % total of the formulation, an emulsifier capable of forming a water-in-oil formulation, one or more skin conditioning agents configured to treat a user's skin, and one or more solvents. The formulation has a minimum viscosity of about 2,000 cPs 24 hours after manufacture. The formulation has a is stable for a minimum time of 8 weeks after continuous exposure to temperature ranging from about 1° C. to about 45° C.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Cosmetic Formulation Properties

The present disclosure relates to cosmetic or dermopharmaceutical compositions containing, for the care of the skin, mucosae and skin appendages, and in some examples for preventing the signs of endogenous and/or exogenous ageing. In an example, the present disclosure provides such as a consumer face.

The formulation includes two polymer components, sodium polyacrylates and xanthan gum, at amounts which provide sufficient formula stability but desirably low viscosity, in conjunction with an emulsifier, among other components. This allows for the formulation to have both a beneficial viscosity and a sheer effect, while remaining relatively stable. The cosmetic formulation discussed herein can be capable of having one or more beneficial effects on a user's skin.

A specific example of a cosmetic formulation according to the instant disclosure is provided below in Table 1.

TABLE 1
Example Formulation.

| | | Approx. |
|---|---|---|
| Component | | Amount (wt %) |
| Solvent | Water | 80-85 |
| Gellant | Sodium polyacrylate | 0.5-0.7 |
| | Xanthan gum | 0.05-0.2 |
| Emulsifier | Potassium cetyl phosphate | 0.4-0.6 |
| Active Ingredients | Bamboo extract | 3.0-3.9 |
| | Thermus thermophilius ferment | |
| | Phellodendron amurense bark | |
| | extract | |
| | Alpha-glucan oligosaccharide | |
| | Kombucha | |
| | Sodium hyaluronate | |
| | Adenosine | |
| Skin Conditioning | Humectants, emollients, and other | 9-15 |
| Agents | | |

| | |
|---|---|
| Preservatives | 0.3-0.5 |
| Fragrance | 0.6 |

Various applications described herein relate to a cosmetic composition that can be used as a topical skin composition. For example, the cosmetic formulation can take the form of a facial cream or serum or a face mask The cosmetic formulation can be useable on a variety of skin types and users. Examples of uses for the cosmetic formulation include use for skin fine lines reduction, improving skin firmness, evening skin tone, strengthening skin barrier, hydrating skin, enhancing skin natural radiance, or a combination thereof.

The cosmetic formulation is in the form of an emulsion. The emulsion typically is an oil-in-water emulsion. In addition to the cosmetic formulation's ability to preserve skin moisture, the cosmetic formulation has additional beneficial properties.

For example, the cosmetic formulation shows commercially acceptable stability. The cosmetic formulation is considered to be stable if it does not phase separate. For example, the cosmetic formulation shows that it is stable for a minimum time of 8 weeks after continuous exposure to temperatures ranging from about 1° C. to about 50° C., about 20° C. to about 45° C., less than, equal to, or greater than about 30° C., 31, 32, 33, 34, 35, 36, 37, 38, 39, 40, 41, 42, 43, 44, 45, 46, 47, 48, 49, or about 50° C. Thus, the cosmetic formulation is able to be stored for a sustained period of time and be usable. Achieving the disclosed stability can be linked to ratios of the thickener to stabilizer as described herein.

Additionally, the cosmetic formulation's viscosity is able to be controlled and remain in a suitable range. For example, a viscosity of the cosmetic formulation measured at both 24 hours after manufacture is in a range of about 2,500 cps to about 3,500 cps, or about 2,700 cps to about 3,300 cps, or about 3,000 cps. The viscosity is measured with a Brookfield RVT 3/20 at a temperature of 25° C. The viscosity of the cosmetic formulation is such that the composition is spreadable but does not run or disperse too quickly. Additionally, the confirmation that the viscosity remains in the aforementioned range after 2 months demonstrates that the viscosity is stable and doesn't go above or below the range quickly. The viscosity can be controlled in part by the disclosed thickener to stabilizer ratios herein.

Moreover, while the stability and viscosity of the cosmetic formulation are beneficial, so is the sheerness, or light-passing property of the formulation. The formulation, thus, when applied, looks beneficial on the skin.

The cosmetic formulation additionally is able to maintain an acceptable pH. For example, when measured following manufacture, a pH of the composition can be in a range of about 5.5 to about 6.5, of about 5.6 to about 6.4, of about 5.7 to about 6.3, of about 5.8 to about 6.2, of about 5.9 to about 6.1, or of about 6.0. The cosmetic formulation's pH can dramatically affect its performance and stability. Therefore, the cosmetic formulation's ability to maintain an acceptable pH over a sufficient amount of time enhances its ability to be a commercially viable product. The pH ranges described herein help to allow for the disclosed thickener to stabilizer ratios to be present.

### Cosmetic Formulation

The aforementioned properties result from the constituents of the cosmetic formulation. While not so limited the cosmetic formulation includes a gellant including two polymers, an emulsifier, solvents, and an active or “skin conditioning” mixture, among other optional components.

### Gellant

The cosmetic formulation includes a gellant (e.g., a thickener) including two polymers: polyacrylates, and xanthan gum. The polymers are provided at amounts specifically to allow for a maximum stability and beneficial viscosity while allowing for sheerness in the formulation and reduced overall polymer content.

The total amount of polymers in the formulation (e.g., the total wt % of the gellant mixture) can remain relatively small, while allowing for beneficial viscosity and stability. For example, the total amount of gellant can be less than about 0.9 wt %, less than about 0.7 wt %, less than about 0.6 wt %, or less than about 0.5 wt % of the total formulation.

The gellant can include two polymers, the first being a sodium polyacrylate. Sodium acrylate copolymer is a sodium salt of a polymer consisting of acrylic acid, methacrylic acid or one of their simple esters. Acrylates are a family of polymers which are a type of vinyl polymer. Synthetic chemicals used in the manufacture of plastics, paint formulations and other products. Acrylate copolymer is a general term for copolymers of two or more monomers consisting of acrylic acid, methacrylic acid or one of their simple esters. Sodium acrylate copolymer is also known as Acrylamide/sodium acrylate copolymer; 2-propenamide, polymer with 2-propenoic acid, sodium salt; 2-propenoic acid, sodium salt, polymer with 2-propenamide; 2-Propenamide-sodium 2 propenoate copolymer; sodium acrylate acrylamide polymer; sodium acrylate-acrylamide copolymer.

The sodium polyacrylate can be provided in an amount of about 0.2 wt % to about 0.6 wt % of the formulation, about 0.25 wt % to about 0.35 wt % of the formulation, or 30 of about 0.3 wt % of the formulation. The gellant can include two polymers, the second being xanthan gum. Xanthan gum is a polysaccharide that is an effective thickening agent, emulsifier, and stabilizer.

The xanthan gum can be provided in an amount of about 0.1 wt % to about 0.2 wt % of the formulation, or about 0.15 wt % of the formulation.

Additional gellants, thickeners, and polymers can be used. For example, the gellant can include any number of additional suitable thickeners to help achieve the desired viscosity values.

Together, the polymers in the gellant can account for less than about 0.9 wt % of the formulation. In the example here, the polyacrylate and the xanthan gum are provided at a ratio of about 6:1 in the formulation.

### Emulsifier

An emulsifier can include any number of components that are capable of forming the water-in-oil composition. In an example, the emulsifier can be Potassium Cetyl Phosphate. It is the potassium salt of a mixture of esters of phosphoric acid and cetyl alcohol, a non-irritating fatty alcohol, that can be used as a stabilizer and emulsifier. The emulsifier can be in a range of about 0.1 wt % to about 1.0 wt % of the formula, about 0.4 wt % to about 0.8 wt % of the composition, or about 0.5 wt % of the composition.

Emulsifiers typically employed in the compositions of the present invention include anionic, nonionic and cationic emulsifiers. See, e.g., Encyclopedia of Chemical Technology, KIRK-OTHMER, volume 22, pp. 333-432, 3rd edition, 1979, Wiley, for the definition of the properties and (emulsifying) functions of the emulsifiers, in particular pp. 347-377 of this publication regarding anionic and nonionic emulsifiers. Examples of emulsifiers useful in the formula of the invention include as nonionic emulsifiers, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic emulsifiers, Ci₆-C₃₀ fatty acids neutralized by amines, ammonia or the alkali metal salts thereof. Examples of cationic emulsifiers include quaternary amines, amine oxides and amines, e.g., alkyl amines, alkyl imidazolines, ethoxylated amines, quaternary compounds, and quaternized esters. Cationic emulsifiers may also provide a conditioning effect. A particularly useful blend of emulsifiers is a blend of ABIL EM 90 and sorbitan olivate. Additional examples of emulsifiers include sodium stearyl glutamate; a mixture of C12-16 alcohols, palmitic acid, and hydrogenated lecithin; hydrogenated coco-glycerides; a mixture of polyglyceryl-6 stearate and polyglyceryl-6 behenate; glyceryl oleate citrate; a mixture of arachidyl alcohol, behenyl alcohol, and arachidyl glucoside; a mixture of ethylhexyl olivate, sodium acrylates copolymer, and polyglyceryl-4 olivate; a mixture of PEG-100 and glyceryl stearate; glyceryl stearate; PEG-2 stearate; or a mixture thereof. An alternative emulsifier can include PEG-40 stearate. PEG-40 stearate is a synthetic polymer composed of PEG (polyethylene glycol) and stearic acid, a naturally occurring fatty acid. It can also be referred to as MACROGOL STEARATE 2000, POLYETHYLENE GLYCOL 2000 MONOSTEARATE, POLYOXYETHYLENE (40) MONOSTEARATE, POLYOXYL 40 STEARATE, and STEARETHATE 40.

The emulsifier is present in the oil or water phase of the emulsion and the gellant is in the water phase of the emulsion.

### Active Agents and Skin Conditioning Agents

The formulation can include one or more active ingredients, which can also act as skin conditioning agents. The active agents includes a mixture of components that act to together on the user's skin. Specifically, the active agents can function to reduce the appearance of wrinkles, hydrate the skin, or the like. For example, the active agents can include Bamboo extract, Thermus thermophilus ferment, Phellodendron amurense bark extract, Alpha-glucan oligosaccharide, Kombucha (Saccharomyces/Xylinum/Black Tea Ferment), Sodium hyaluronate, and Adenosine.

Bamboo extract (Phyllostachys bambusoides Extract) is a can be used for invigorating face and body lotion. Bamboo extract can be in a range of about 0.80 wt % to about 1.20 wt % of the cosmetic composition, about 0.90 wt % to about 1.10 wt %, or about than about 1.0 wt %.

Thermus thermophilus ferment is the natural result from the fermentation of Thermus thermophilus, a bacterium that can live thousands of feet below the ocean's surface. On skin, this special ferment can help protect skin's support structure from falling prey to destructive enzymes and help maintain a firm feel. It also has antioxidant ability, particularly on rogue molecules formed as fatty acids break down. The radical-scavenging ability of Thermus thermophilus has been compared to potent antioxidants in skin such as superoxide dismutase. It's a very good source of alpha-galactosidase, an enzyme which helps replenish glycoproteins, which skin needs to look healthier. Thermus thermophilus ferment can be used, for example, mixed with glycerin. The mixture of Thermus thermophilus ferment and glycerin can be in a range of about 0.50 wt % to about 1.50 wt % of the cosmetic composition, about 0.80 wt % to about 1.20 wt %, or about 1.0 wt %.

Phellodendron amurense bark extract can have anti-inflammatory, anti-oxidant and potential chemopreventive and antineoplastic activities. Phellodendron amurense bark extract contains certain isoquinoline alkaloids, flavone glycosides and phenolic compounds. Phellodendron amurense bark extract can be provided mixed with water and butylene glycol in an amount of about 0.40 wt % to about 0.80 wt % of the cosmetic composition, about 0.50 wt % to about 0.70 wt %, or about 0.6 wt %.

α-glucan oligosaccharide is a natural prebiotic that helps support a healthy microbiome. When skin's microbiome (it's invisible barrier) is disrupted, the population of harmful bacteria can grow since it's not being kept in check by healthy flora on the surface. This has the potential to trigger signs of irritation and sensitized skin. α-glucan oligosaccharide also has antioxidant benefit, helping to reduce the visible effects of oxidative stress on skin. α-glucan oligosaccharide can be in a range of about 0.3 wt % to about 0.7 wt % of the cosmetic composition, about 0.4 wt % to about 0.6 wt %, or about 0.5 wt %.

Kombucha (also tea mushroom, tea fungus, or Manchurian mushroom; Latin name Medusomyces gisevii, e.g., Saccharomyces/Xylinum/Black Tea Ferment) is a fermented, lightly effervescent, sweetened black tea drink. For example, Kombucha can be used in the formulation, such as includes skin conditioners such as Saccharomyces, xylinum, black tea ferment, and glycerin, among other components. In an example formulation, Kombucha is in a range of about 0.3 wt % to about 0.8 wt % of the cosmetic composition, about 0.4 wt % to about 0.6 wt %, or about 0.5 wt %.

Sodium hyaluronate is a derivative of hyaluronic acid. It has many uses, and is a humectant, which means that it attracts moisture. Sodium hyaluronate can be used in a range of about 0.08 wt % to about 0.12 wt %, of about 0.09 wt % to about 0.11 wt %, or about 0.1 wt %. Adenosine can be a skin conditioning agent and active component. Adenosine can be used in a range of about 0.03 wt % to about 0.05 wt %, or at about 0.04 wt %.

The active agents can be located in the water phase. The formulation can additionally include one or more other skin conditioning agents, such as emollients, humectants, other conditioning agents, and combinations thereof.

Additionally skin conditioning agents can include emollients and humectants. Emollients can have the quality of softening or smoothing the skin. Example emollients can include caprylic/capric triglyceride, coco-caprylate/caparate, caprylyl glycol, or combinations thereof. Humectants can aid in retaining moisture in formulation and moisturizing skin when applied. Example humectants can include glycerin, and combinations thereof. Other skin conditioning agents, such as occlusive agents, can be used, for example, dimethicone or limnanthes alba (meadowfoam) seed oil.

A variety of other skin conditioners can be included to achieve various skin treatment effects. Additional examples of such active skin condition agents can include lactic acid, a hyaluronic acid, vitamin E, Saccharomyces/xylinum/black tea ferment, diglucosyl gallic acid, adenosine, panthenol, dimethylmethoxy chromanol, alpha-glucan oligosaccharide, Thermus thermophilus ferment, hydrolyzed hyaluronic acid, caffeine, acetyl di-peptide-1 cetyl ester, glycerin, niacinamide, swertia chirata extract, creatine, or a mixture thereof.

The total active and skin conditioning agents can be in a range of about 5 wt % to about 35 wt % of the cosmetic formula, about 20 wt % to about 30 wt %, less than, equal to, or greater than 15 wt %, 16, 17, 18, 19, 20, 21, 22, 23, 24, 25, 26, 27, 28, 29, 30, 31, 32, 33, 34, or about 35 wt %.

The active agents and skin conditioning agents can include any component or mixture of components to achieve a desired result. The gellant mixture discussed herein above, allow for the relatively high number of active agents and skin conditioning agents. The ability of the formulation to have a high number of active and skin conditioning agents can enhance the formulation's effectiveness.

### Solvents

The formulation can additionally include one or more solvents. For example, the formulation can include water, such as for the aqueous phase, that may form an emulsion with the oil based phase. Other solvents may be used as desired, such as 1,2-hexanediol or butylene glycol, among other organic solvents.

### Other Components

The formulas herein can contain a variety of other optional components suitable for rendering such compositions more cosmetically or aesthetically acceptable or to provide them with additional usage benefits. For example, the formulation can additionally include a variety of other components, such as pH adjusters, preservatives, and fragrance. For example, an appropriate pH adjuster can include sodium hydroxide. For example, potential preservatives can include chlorphenesin, potassium sorbate, sodium benzoate, or other components.

Such conventional optional ingredients are well-known to those skilled in the art. These include any cosmetically acceptable ingredients such as those found in the CTFA International Cosmetic Ingredient Dictionary and Handbook, 7th edition, edited by Wenninger and McEwen, (The Cosmetic, Toiletry, and Fragrance Association, Inc., Washington, D.C., 1997). As used herein “cosmetically acceptable” means a material (e.g., compound or composition) which is suitable for use in contact with skin, hair or other suitable substrate.

### Method of Making

The cosmetic formulations described herein can be produced according to many suitable methods. The cosmetic formulation can be in the form of an emulsion, having a water phase and an oil phase. The water phase, which can be mixed together, can include solvents, active agents, and some of the skin conditioning agents. The oil phase can include the emulsifier, in addition to other skin conditioning agents. The water phase and the oil phase can be mixed together for period of time to form the emulsion cosmetic composition. Additional components, such as the fragrance, can be added at the end. All the components can be stirred or otherwise mixed to form the final cosmetic formulation emulsion.

### Method of Using

The cosmetic formulation can be applied in any suitable manner. For example the cosmetic formulation can be topically applied to a user's skin. In some cases, the cosmetic composition can be topically applied by hand, with an applicator, or both. Examples of applicators can include brushes, pads, sponges, or the like. In some cases, the cosmetic formulation can be used as a serum, a cream, or a mask. In some cases, a silicone mask can be used for application.

The cosmetic formulation can be applied once daily (e.g., once every 24 hours) or multiple times throughout the day. For example, the cosmetic formula can be applied two times, three times, four times, and so on within a 24 hour period.

### EXAMPLES

Various examples of the present disclosure can be better understood by reference to the following Examples which are offered by way of illustration. The present disclosure is not limited to the Examples given herein.

A sample formulation was made according to the below Table 2:

TABLE 2
Sample formulation.

| Trade Name | Vendor | Raw Material % |
|---|---|---|
| Water Purified | COTY | 82.16% |
| GLYCERINE G995E | AMI | 5% |
| Dub 5545 14012 MB | STEARINERIE DUBOIS | 3% |
| RBD Meadowfoam Oil | KEYSER & MACKAY | 2% |
| Cetiol LC (30527919) | BASF | 1% |
| Original bamboo extract | GATTEFOSSE | 1% |
| VENUCEANE SP | SEDERMA | 1% |
| Cosmedia SP (30531250) | BASF | 0.6% |
| OUBAKU LIQUID B | Safic-Alcan | 0.6% |
| LIEN DE LIERRE NABI | FIRMENICH | 0.6% |
| 297136 | | |
| AMPHISOL K | DSM NUTRITIONAL | 0.5% |
| | PRODUCTS | |
| BioEcolia (AC033) | SOLABIA | 0.5% |
| NG KOMBUCHKA | SEDERMA | 0.5% |
| Xiameter PMX-200 | DOW | 0.5% |
| Silicone Fluid 100cs | | |
| Elastab CPN | BASF/IMCD | 0.3% |
| (30531050)/DEKA CP | | |
| 266960 | | |
| SymDiol 68 | SYMRISE | 0.3% |
| Sensiva SC 50 | ASHLAND | 0.2% |
| Rhodicare S | SACI | 0.1% |
| CristalHyal | GIVAUDAN | 0.1% |
| Adenosine (A051) | DKSH | 0.04% |

The components of this sample formulation were mixed together. This sample was tested for viscosity, pH, and stability as outlined below in Example 1 and Example 2.

### Example 1 Viscosity and pH of Sample Formulation

The Sample summarized in Table 3 was tested for viscosity. The viscosity was measured with a Brookfield RVT E/20 at a temperature of 25° C., using Spindle 3, a speed of 20 rpm, and a multiplier of 50, with the results shown in Table 3. The samples were tested with standard pH strips to determine pH, also summarized in Table 3.

TABLE 3
Viscosity & pH Testing

| Test No. | Viscosity (cps) | pH |
|---|---|---|
| 1 | 2,325 | 5.96 |
| 2 | 3,100 | 5.90 |
| 3 | 2,910 | 5.92 |
| 4 | 2,950 | 5.96 |
| 5 | 2,800 | 5.90 |
| 6 | 2,850 | 5.92 |
| 7 | 2,875 | 5.81 |
| 8 | 2,795 | 6.02 |
| 9 | 2,750 | 5.99 |
| 10 | 2,815 | 5.96 |
| 11 | 2,820 | 5.79 |

Overall, the Sample performed well with both viscosity and pH, despite the lower overall amount of gellant in the formulation.

### Example 2. Stability of Sample Formulation

The Sample was manually analyzed for stability, color, and odor, at 24 hours after manufacture, and at subsequent weeks and months after manufacture. The results are summarized below in Table 4:

TABLE 4
Manual Review of Sample

| | | Room | | | |
|---|---|---|---|---|---|
| | Fridge | Temp | | | |
| | (4° C.) | 25° C. | 37° C. | 45° C. | 50° C. |
| Color | 4 weeks | 4 weeks | 2 months | 3 months | 4 weeks |
| | Pass | Pass | Pass | Pass | Pass |
| Odor | 4 weeks | 4 weeks | 2 months | 3 months | 4 weeks |
| | Pass | Pass | Pass | Pass | Pass |
| Stability | 4 weeks | 4 weeks | 2 months | 3 months | 4 weeks |
| | Pass | Pass | Pass | Pass | Pass |

As can been seen in Table 4, the formulation sample showed good stability over a range of temperatures, from fridge (1-4° C.) to room temperature (20-22° C.) up to 50° C. immediately after manufacture and up to four weeks after manufacture. The formulation still maintained good stability at up to about 50° C.

### Additional Examples

The following exemplary examples are provided, the numbering of which is not to be construed as designating levels of importance:

Example 1 includes a cosmetic formulation comprising: a gellant comprising a sodium polyacrylate and xanthan gum, wherein the gellant comprises less than about 0.8 wt % total of the formulation; an emulsifier capable of forming a water-in-oil formulation; one or more active agents configured to treat a user's skin; and one or more solvents; wherein the formulation has a minimum viscosity of about 2,500 cPs 24 hours after manufacture, and wherein the formulation has a is stable for a minimum time of 4 weeks after continuous exposure to temperature ranging from about 1° C. to about 50° C.

Example 2 can include Example 1, wherein the sodium polyacrylate is about 0.2 wt % to about 0.7 wt % of the formulation.

Example 3 can include any of Examples 1-2, wherein the sodium polyacrylate is about 0.3 wt % to about 0.6 wt % of the formulation.

Example 4 can include any of Examples 1-3, wherein the sodium polyacrylate is about 0.6 wt % of the formulation.

Example 5 can include any of Examples 1-1-4, wherein the xanthan gum is about 0.05 wt % to about 0.15 wt % of the formulation.

Example 6 can include any of Examples 1-5, wherein the xanthan gum is about 0.1 wt % of the formulation.

Example 7 can include any of Examples 1-1, wherein the sodium polyacrylate and the xanthan gum are provided at a ratio of about 6:1 in the formulation.

Example 8 can include any of Examples 1-1-7, wherein the formulation has a minimum viscosity of about 3,000 cPs.

Example 9 can include any of Examples 1-8, wherein the formulation has a minimum viscosity of about 3,500 cPs.

Example 10 can include any of Examples 1-1-9, wherein the formulation is stable for a minimum time of 4 weeks after continuous exposure to temperature ranging from about 25° C. to about 45° C.

Example 11 can include any of Examples 1-10, wherein the formulation is stable for a minimum time of 4 weeks after continuous exposure to temperature of about 37° C.

Example 12 can include any of Examples 1-1-11, wherein the formulation is stable for a minimum time of 8 weeks after continuous exposure to temperature ranging from about 1° C. to about 50° C.

Example 13 can include any of Examples 1-12, wherein the formulation is stable for a minimum time of 8 weeks after continuous exposure to temperature ranging from about 20° C. to about 45° C.

Example 14 can include any of Examples 1-1-13, wherein the emulsifier is about 0.10 wt % to about 1.0 wt % of the formulation.

Example 15 can include any of Examples 1-1-14, wherein the emulsifier comprises potassium cetyl phosphate.

Example 16 can include any of Examples 1-1-15, wherein the active agents comprises Bamboo extract, Thermus thermophilus ferment, Phellodendron amurense bark extract, alpha-glucan oligosaccharide, kombucha, sodium hyaluronate, adenosine, and combinations thereof.

Example 17 can include any of Examples 1-1-16, further comprising one or more skin conditioning agents comprising caprylic/capric triglyceride, coco-caprylate/caparate, caprylyl glycol, glycerin, or combinations thereof.

Example 18 can include any of Examples 1-1-17, further comprising one or more preservatives, buffers, fragrances, pigments, or combinations thereof.

Example 19 includes a method of making the formulation of any of claims 1-18, the method comprising mixing the one or more solvents, the emulsifier, the gellant, and the one or more skin conditioning agents to form the cosmetic composition.

Example 20 includes a method of treating skin, the method comprising topically applying formulation of any one of claims 1-19 to a person's skin, wherein the formulation brightens the skin.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed may be resorted to by those of ordinary skill in the art, and that such modifications and variations are considered to be within the scope of examples of the present disclosure.

1. A cosmetic formulation comprising:
a gellant comprising a sodium polyacrylate and xanthan gum, wherein the gellant comprises less than about 0.9 wt % total of the formulation;
between about 0.10 wt % to about 1.0 wt % of an emulsifier capable of forming a water-in-oil formulation, wherein the emulsifier is potassium cetyl phosphate;
one or more active agents configured to treat a user's skin; and
one or more solvents;
wherein the formulation has a minimum viscosity of about 2,500 cPs 24 hours after manufacture, and
wherein the formulation is stable for a minimum time of 4 weeks after continuous exposure to temperature ranging from about 1° C. to about 50° C.

2. The formulation of claim 1, wherein the sodium polyacrylate is about 0.2 wt % to about 0.7 wt % of the formulation.

3. The formulation of claim 2, wherein the sodium polyacrylate is about 0.3 wt % to about 0.6 wt % of the formulation.

4. The formulation of claim 3, wherein the sodium polyacrylate is about 0.6 wt % of the formulation.

5. The formulation of claim 1, wherein the xanthan gum is about 0.05 wt % to about 0.15 wt % of the formulation.

6. The formulation of claim 5, wherein the xanthan gum is about 0.1 wt % of the formulation.

7. The formulation of claim 1, wherein the sodium polyacrylate and the xanthan gum are provided at a ratio of about 6:1 in the formulation.

8. The formulation of claim 1, wherein the formulation has a minimum viscosity of about 3,000 cPs.

9. The formulation of claim 8, wherein the formulation has a minimum viscosity of about 3,500 cPs.

10. The formulation of claim 1, wherein the formulation is stable for a minimum time of 4 weeks after continuous exposure to temperature ranging from about 25° C. to about 45° C.

11. The formulation of claim 10, wherein the formulation is stable for a minimum time of 4 weeks after continuous exposure to temperature of about 37° C.

12. The formulation of claim 1, wherein the formulation is stable for a minimum time of 8 weeks after continuous exposure to temperature ranging from about 1° C. to about 50° C.

13. The formulation of claim 12, wherein the formulation is stable for a minimum time of 8 weeks after continuous exposure to temperature ranging from about 20° C. to about 45° C.

14. The formulation of claim 1, wherein the active agents comprises Bamboo extract, Thermus thermophilus ferment, Phellodendron amurense bark extract, alpha-glucan oligosaccharide, Saccharomyces/xylinum/black tea ferment, sodium hyaluronate, adenosine, and combinations thereof.

15. The formulation of claim 1, further comprising one or more skin conditioning agents comprising caprylic/capric triglyceride, coco-caprylate/caparate, caprylyl glycol, glycerin, or combinations thereof.

16. The formulation of claim 1, further comprising one or more preservatives, buffers, fragrances, pigments, or combinations thereof.

17. A method of making the formulation of claim 1, the method comprising mixing the one or more solvents, the emulsifier, the gellant, and the one or more skin conditioning agents to form the cosmetic composition.

18. A method of treating skin, the method comprising topically applying formulation of claim 1 to a person's skin, wherein the formulation moisturizes the skin.
